(19) United States
(12) Patent Application Publication
(10) Pub. No.: US 2011/0039765 A1
(43) Pub. Date: Feb. 17, 2011

(54) Bioactive Molecules Produced by Probiotic Bacteria

(76) Inventor: Ruth I. Connor, Hartland, VT (US)

Correspondence Address: LICATA & TYRRELL P.C., 66 E. MAIN STREET, MARLTON, NJ, 08053, US

(21) Appl. No.: 12/937,417

(22) PCT Filed: Mar. 9, 2009
(86) PCT No.: PCT/US2009/036451

§ 371 Date: Oct. 12, 2010

Related U.S. Application Data
(60) Provisional application No. 61/045,779, filed on Apr. 17, 2008.

(51) Int. Cl. A61K 38/02; C07K 1/20; C07K 2/00; C12N 5/071; A61P 31/18

(52) U.S. Cl. 514/3.8; 530/344; 530/300; 435/375

(57) ABSTRACT

The present invention is a method for isolating bioactive molecules secreted by probiotic bacteria such as Lactobacillus rhamnosus, and methods for using such bioactive molecules to decrease replication of human immunodeficiency virus, expression of inflammatory cytokines and chemokines, expression of vasoendothelial growth factor, Erk1/Erk2 activation, and to inhibit HIV transmission.

This application claims benefit of priority from U.S. Provisional Patent Application Ser. No. 61/045,779 filed Apr. 17, 2008, the content of which is incorporated herein by reference in its entirety.

This invention was made with government support under Grant Nos. R21AI065235-02 and 1R21AI071948-01A1 awarded by the National Institutes of Health. The government has certain rights in the invention.

### BACKGROUND OF THE INVENTION

Probiotics are live microorganisms which, when administered in adequate amounts, confer a health benefit to the host. Probiotics most commonly include strains of lactic acid bacteria within the genera of Lactobacillus and Bifidobacteria. In clinical trials, ingestion of live probiotic bacteria is associated with improvement in intestinal and immune health when probiotic products are consumed on a regular basis.

Health benefits conferred by probiotics are presumed to be dependent on maintenance and delivery of viable bacteria in adequate doses. However, live probiotics are subject to loss of viability and compromised quality due to exposure of the bacteria to moisture, heat, and changes in pH during manufacturing and storage. These conditions limit the inclusion of probiotic organisms in consumer products and therapeutics.

Culture filtrate from probiotic bacteria has been suggested for use in the treatment of disease. In this regard, Japanese Patent number JP63316726 suggests a polysaccharide or a protein polysaccharide produced by a bacterium belonging to Procaryomycota as an antiviral agent for suppressing infection of a retrovirus.

U.S. patent application Ser. No. 10/878,411 teaches a method of treating a mammal having a retroviral infection by administering a therapeutic agent composed of a concentrate of a filtrate of a culture solution obtained by co-culturing a particular combination of different types of beneficial organisms selected from yeasts and lactic acid bacteria.

U.S. patent application Ser. No. 10/413,993 teaches a method of preventing and treating viral infections or inhibiting the spread of viruses by administering compositions of at least one Lactobacillus whole cell or by-product thereof to patients in need of such treatment.

U.S. patent application Ser. No. 10/831,070 teaches L. acidophilus cell wall, cell surface and secreted proteins and fragments and variants thereof for use in preventing or reducing the occurrence of an infection in a host.

Silva, et al. (1987) Antimicrob. Agents Chemother. 31(8):1231-3) teach the presence of a low molecular weight compound (<1000 Da) secreted by LGG, that is heat stable, active between pH 3-5, distinct from lactic and acetic acids, and soluble in acetone water. It is suggested that this compound may be a short-chain fatty acid or microcin.

Tao, et al. (2006) Am. J. Physiol. Cell Physiol. 290(4):C1018-30) teach that soluble factors produced by LGG activate MAPK and induce heat shock proteins in intestinal epithelial cells. These effects are mediated by a low-molecular weight peptide that is heat and acid stable, protease sensitive, and <10 kDa.

### SUMMARY OF THE INVENTION

The present invention is a method for isolating a bioactive molecule from a probiotic bacterium, e.g., from the genera Lactobacillus, in particular L. rhamnosus. The method involves passing probiotic bacterium-conditioned media through a reverse-phase chromatography matrix to obtain hydrophilic molecules having a molecular weight of less than 1000 Daltons; and further fractionating the molecules by size exclusion to isolate a bioactive molecule characterized as being hydrophilic; stable to at least 99° C., stable at pH from about 3.0 to about 10.0, resistant to protease digestion, and having a molecular weight of between about 500 Daltons and about 700 Daltons, wherein said isolated bioactive molecule inhibits replication of human immunodeficiency virus, decreases expression of inflammatory cytokines and chemokines, decreases expression of vasoendothelial growth factor, and decreases Erk1/Erk2 activation.

An isolated bioactive molecule from a probiotic bacterium is also provided as are methods for using the same for inhibiting replication of human immunodeficiency virus, decreasing expression of inflammatory cytokines and chemokines, decreasing expression of vasoendothelial growth factor, decreasing extracellular signal-regulated kinases 1/2 activation. In addition, a pharmaceutical composition including the isolated bioactive molecule in admixture with a pharmaceutically acceptable carrier is disclosed for use in preventing mucosal transmission of HIV.

### DETAILED DESCRIPTION OF THE INVENTION

Culture and fractionation methods have now been found that yield small bioactive molecules (500-700 Da) secreted by the lactic acid bacterium Lactobacillus rhamnosus GG (LGG). The molecules are hydrophilic, heat stable, acid stable, and resistant to proteolytic enzymes. Furthermore, the molecules decrease HIV replication in susceptible target cells and tissues, decrease secretion of inflammatory immune mediators (e.g., IL-1ra, IL-6, and IL-8), decrease secretion of vasoendothelial growth factor (VEGF), and decrease activation of cell signaling through MAPK (Erk1/Erk2; extracellular signal-regulated kinases 1/2) pathways. Given their potent activity, these molecules find application as topical microbicides for preventing HIV transmission in both adults and infants. For example, the bioactive molecules herein can be formulated in a topical microbicide for vaginal or rectal use to prevent HIV sexual transmission or as a topical or oral formulation for use in infants exposed to HIV through breastfeeding.

Bioactive molecules of the present invention are isolated by passing probiotic bacterium-conditioned medium through a reverse-phase chromatography matrix to obtain hydrophilic molecules having a molecular weight of less than 1000 Daltons; and further fractionating the flow-through from the reverse-phase chromatography matrix by size exclusion. Size exclusion fractions embraced by the present invention contain bioactive molecules characterized as being hydrophilic; stable to at least 99° C.; stable at pH from about 3.0 to about 10.0; resistant to protease digestion; and as having a molecular weight of between about 500 Daltons and about 700 Daltons. In addition to these physical characteristics, the bioactive molecules of the invention have a variety of biological activities.

As used herein, probiotic bacteria are live microorganisms which, when administered in adequate amounts, confer a health benefit to the host. Lactic acid bacteria are the most common type of probiotic bacteria. For the purposes of the present invention, lactic acid bacteria include bacteria of the genera Lactobacillus (e.g., L. rhamnosus, L. acidophilus, L. jensenii, L. plantarum, L. gasseri and L. crispatus), Streptococcus (e.g., S. thermophilus and S. salivarius), Enterococcus (e.g., E. faecalis) and Bifidobacterium (e.g., B. animalis, B. breve, B. infantis, B. lactis, and B. longum). Having demonstrated the presence of bioactive molecules in the culture medium of bacteria from each of these genera, particular embodiments of the invention embrace the use of lactic acid bacteria for obtaining the bioactive molecules of the invention. In certain embodiments, the probiotic bacterium is from the genus Lactobacillus. In particular embodiments, the probiotic bacterium is L. rhamnosus.

To provide a high yield of bioactive molecules in the culture medium, the probiotic bacterium is grown at 37° C. under either anaerobic or aerobic conditions, wherein the cultures are initiated in media at, or near, neutral pH (i.e., 6.0 to 8.0) without supplemental CO₂. After growing the probiotic bacterium for a sufficient amount of time to produce the bioactive molecules, e.g., at least eight to 24 hours, the culture medium is isolated from the probiotic bacterium (e.g., by filtration or centrifugation) and the culture medium is fractionated. In so far as the culture medium contains bioactive molecules produced or secreted by the probiotic bacterium, the culture medium is set forth herein as “probiotic bacterium-conditioned medium.”

Reverse-phase chromatography includes any chromatographic method that uses a non-polar stationary phase. Samples applied to a reverse-phase chromatography matrix are separated based on the principle of partitioning between the mobile and stationary liquid phases and can be carried out by means of either a high-performance liquid chromatography column or a fast protein liquid chromatography column. In general, the mobile phase encompasses two solvent solutions, a polar and a non-polar solvent, to be blended via a gradient over the course of the chromatographic separation. Upon application of probiotic bacterium-conditioned media to a reverse-phase chromatography matrix, the initial flow-through is collected. This flow-through contains salts, hydrophilic amino acids and small hydrophilic peptides of less than 1000 Daltons. It is in this initial flow-through that the bioactive molecules of the present invention are found. Reverse-phase matrices suitable for purification of bioactive molecules of this invention include columns packed with silica beads bearing alkyl groups ranging in length from 4-18 carbon atoms, i.e., C₄-C₁₈. In particular embodiments, the reverse-phase chromatography matrix is a C₁₈ matrix. Reagents and methods for carrying our reverse-phase chromatography are routinely practiced in the art. See, e.g., Scopes, et al. (January 1994) In: Protein Purification: Principles and Practice, 3rd edition, Springer Verlag.

Flow-through collected from the reverse-phase chromatography matrix is subsequently applied to a size exclusion column to elute and isolate fractions containing hydrophilic bioactive molecules of 500 Dalton to 700 Dalton in size. Any suitable size exclusion method can be used including, e.g., gel filtration chromatography, gel permeation chromatography, or gel electrophoresis. The matrix for size exclusion can be polyacrylamide, dextran, agarose, silica or crosslinked polystyrene. Commonly employed size exclusion matrices include SUPERDEX-, SEPHADEX-, SUPEROSE-, SEPHAROSE- and SEPHACRYL-based matrices. Fractions containing the bioactive molecules of the invention can be identified based upon the presence of molecules having one or more of the biophysical characteristics or biological activities of the bioactive molecules of the invention. Analysis of fractions can be carried out as described herein or using any other conventional method for determining such biophysical characteristics or biological activities.

It is contemplated the bioactive molecules of the invention can be used as a fraction obtained from a size exclusion matrix or alternatively be further purified to homogeneity. Methods for purifying one or more bioactive molecules to homogeneity (e.g., greater than 90%, 95%, or 99% purity) include thin layer chromatography, SDS-PAGE, ion exchange HPLC, and the like. Such methods are routinely employed in the art to purify molecules of interest and any one or combination of methods can be employed.

The bioactive molecules of the invention are isolated in the sense that the bioactive molecules have been removed from their natural environment, i.e., probiotic bacterium-conditioned medium, in a form to achieve a significant increase in activity over crude extracts having said bioactive molecules. Such isolated bioactive molecules can include, but are not limited to, bioactive molecules purified to homogeneity, recombinantly produced bioactive molecules, and isolated bioactive molecules which have been fractionated by column chromatography (e.g., reverse-phase and size exclusion) and may contain other peptides or amino acids.

The bioactive molecules of the present invention can include, but are not limited to, small peptides that are heat and acid stable, and demonstrate significant ability to modulate cellular inflammatory responses and inhibit HIV replication. In so far as the bioactive molecules are stable and biologically active under a variety of conditions, these molecules are particularly useful, alone or in pharmaceutical compositions, for inhibiting HIV replication, decreasing expression of inflammatory cytokines and chemokines, decreasing VEGF secretion, decreasing Erk1/Erk2 activation and preventing mucosal transmission of HIV in vitro or in vivo.

Accordingly, the present invention further provides the use of the bioactive molecules of the invention for inhibiting HIV replication, decreasing expression of inflammatory cytokines and chemokines, decreasing expression of VEGF and decreasing activation of Erk1/Erk2 in a cell. Cells of particular application in accordance with such methods include human epithelial cells and cells of primary tissues which contain epithelial cells and CD4+ cells. Such cells are of particular interest in so far as they are located at mucosal surfaces and are in direct contact with or susceptible to infection by pathogens such as HIV. In this regard, some embodiments of the invention provide for cells already infected with HIV, as well as cells which are free of HIV. In carrying out these methods, a cell is contacted with an effective amount of one or more isolated bioactive molecules of the invention such that a detectable decrease in HIV replication (e.g., as determined by transcription of HIV), inflammatory cytokine and chemokine expression (e.g., IL-1ra, IL-6 or IL-8), VEGF expression, or Erk1/Erk2 activation is achieved as compared to a cell not contacted with the isolated bioactive molecules. A detectable decrease includes at least a 20%, 30%, 40%, 50%, 60%, 70%, 80%, 90%, or 100% decrease as determined using the methods disclosed here or any other suitable methods routinely practiced in the art for detecting HIV replication, inflammatory cytokine and chemokine expression, VEGF expression, or Erk1/Erk2 activation.

In so far as VEGF, Erk1/Erk2, and proinflammatory cytokine and chemokines such as IL-6, IL-1ra, and IL-8 are involved in a variety of diseases and conditions besides HIV infection, the disclosed methods of the invention also find application in the prevention and treatment of such diseases and conditions. For example, increased IL-8 expression is associated with poor clinical outcome in human ovarian carcinoma, and decreasing IL-8 expression has been shown to decrease tumor growth through antiangiogenic mechanisms (Merritt, et al. (2008) J. Natl. Cancer Inst. 100(5):359-72). Similarly, inhibition of VEGF expression has been shown to inhibit the growth of colorectal cancer (Lv, et al. (2007) Cancer Biother. Radiopharm. 22(6):841-52). In addition, inhibition of the Erk pathway has been shown to prevent HIVgp120-induced REM sleep increase (Diaz-Ruiz, et al. (2001) Brain Res. 913:78-81). Accordingly, the bioactive molecules of the invention can be used in the treatment of diseases and conditions including, but not limited to, autoimmune diseases, cancer, infections, as well as tissue damage due to inflammatory responses.

While a direct interaction with HIV was not identified, the bioactive molecules of the invention were shown to decrease activation of host cell pathways, which regulate HIV transcription and therefore HIV replication and transmission. Accordingly, the present invention further embraces a method for preventing mucosal transmission of HIV by administering to the mucosa of a subject an effective amount of the bioactive molecules of the invention, or a pharmaceutical composition containing such bioactive molecules. In particular embodiments, the bioactive molecules are topically applied in an acid-buffering gel or cream to prevent mucosal transmission of HIV-1 in the female reproductive tract, or with a neutral pH gel or cream to prevent rectal transmission of HIV-1 in adults.

Bioactive molecules of the present invention can be conveniently used or administered in a composition containing one or more active molecules in combination with a pharmaceutically acceptable carrier. Such compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A carrier, pharmaceutically acceptable carrier, or vehicle, such as a liquid or solid filler, diluent, excipient, or solvent encapsulating material, is involved in carrying or transporting the subject active molecules from one organ, or portion of the body, to another organ, or portion of the body. Each carrier must be acceptable in the sense of being compatible with the other ingredients of the formulation and not injurious to the patient.

Examples of materials which can serve as carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Bioactive molecules of the invention can be administered via any route including, but not limited to, oral, rectal, buccal (e.g., sub-lingual), vaginal, parenteral (e.g., subcutaneous, intramuscular, intradermal, intravenous, intraperitoneal), topical (i.e., both skin and mucosal surfaces, including vaginal and gastrointestinal surfaces), intranasal, transdermal, intraarticular, intrathecal and inhalation administration. The most suitable route in any given case will depend on the nature and severity of the condition being prevented or treated and on the nature of the particular bioactive molecule(s) being used.

For injection, the carrier will typically be a liquid, such as sterile pyrogen-free water, pyrogen-free phosphate-buffered saline solution, bacteriostatic water, or CREMOPHOR (BASF, Parsippany, N.J.). For other methods of administration, the carrier can be either solid or liquid.

For oral administration, the compound can be combined with one or more carriers and used in the form of ingestible tablets, buccal tablets, troches, capsules, elixirs, suspensions, syrups, wafers, chewing gums, foods and the like. Such compositions and preparations typically contain at least 0.1% of bioactive molecule(s). The percentage of the bioactive molecule(s) and preparations can, of course, be varied and can conveniently be between about 0.1 to about 100% of the weight of a given unit dosage form. The amount of bioactive molecule(s) in such compositions is such that an effective dosage level will be obtained. In particular embodiments, the bioactive molecules are formulated in an oral composition for use in preventing HIV-1 transmission in the gastrointestinal tract of infants exposed to HIV-1 through breastfeeding. The isolated bioactive molecules can be combined with a neutral pH agent (e.g., a gel, cream or infant formula) for oral application.

When prepared in the form of tablets, troches, pills, capsules, and the like, such formulations can also contain binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

A syrup or elixir can contain the active agent, sucrose or fructose as a sweetening agent, methyl and propylparabens as preservatives, a dye and flavoring such as cherry or orange flavor. Of course, any material used in preparing any unit dosage form should be substantially non-toxic in the amounts employed. In addition, the active compounds can be incorporated into sustained-release preparations and devices including, but not limited to, those relying on osmotic pressures to obtain a desired release profile.

Formulations of the present invention suitable for parenteral administration contain sterile aqueous and non-aqueous injection solutions of the bioactive molecules, which preparations are generally isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution of the compound. Suitable formulations contain citrate or Bis\Tris buffer (pH 6) or ethanol/water and contain from 0.1 to 0.2 M of the compound.

A compound can alternatively be formulated for nasal administration or otherwise administered to the lungs of a subject by any suitable means. In particular embodiments, the compound is administered by an aerosol suspension of respirable particles containing the bioactive molecule(s), which the subject inhales. The respirable particles can be liquid or solid. The term aerosol includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn, et al. (1992) J. Pharmacol. Toxicol. Methods 27:143-159. Aerosols of liquid particles containing the bioactive molecule(s) can be produced by any suitable means, such as with a pressure-driven aerosol nebulizer or an ultrasonic nebulizer, as is known to those of skill in the art. See, e.g., U.S. Pat. No. 4,501,729. Aerosols of solid particles containing the bioactive molecule(s) can likewise be produced with any solid particulate medicament aerosol generator, by techniques known in the pharmaceutical art.

Formulations suitable for topical application to the skin can take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof. In particular, bioactive molecules of the invention can be formulated with specific acid-buffering agents, in the form of gels or creams, designed for topical use. Acid-buffering products that maintain vaginal fluid at a mildly acidic pH (4.5-5.0) are conventionally employed in clinical trials as topical microbicides to prevent HIV-1 infection (e.g., BUFFERGEL™; ReProtect Inc., Baltimore, Md.). Accordingly, particular embodiments of the invention embrace a pharmaceutical composition containing the isolated bioactive small molecules with an acid-buffering gel or cream for preventing HIV-1 infection across mucosal surfaces in the vagina. In alternative embodiments, formulation of the bioactive molecules with a neutral pH gel or cream can be used for rectal use against HIV-1 sexual transmission.

In particular embodiments, the bioactive molecules of the invention are administered to a subject in an effective amount. Dosages of bioactive molecules can be determined by methods known in the art, see, e.g., Remington: The Science and Practice of Pharmacy, supra. The selected effective dosage level will depend upon a variety of factors including the activity of the particular bioactive molecule(s) employed, the route of administration, the time of administration, the rate of excretion or metabolism of the particular bioactive molecule(s) being employed, the duration of the treatment, other drugs, compounds and/or materials used in combination with the particular bioactive molecule(s) employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required for prevention or treatment in a subject (e.g., a human) based upon clinical and preclinical trials of the pharmaceutical composition for the disease or condition being prevented or treated.

Such preclinical trials can include the evaluation of toxicity and cytopathology following topical application of the bioactive molecules directly to vaginal tissues in a murine model. In addition, preclinical data can be obtained regarding the impact of body fluids, namely vaginal secretions and human serum, on the bioactivity of the isolated bioactive molecules disclosed herein.

The invention is described in greater detail by the following non-limiting examples.

### EXAMPLE 1

### Isolation of Bioactive Small Molecules from Probiotic Bacteria

Bacterial Culture: The Lactobacillus rhamnosus GG (LGG) strain was obtained from ATCC (Manassas, Va.) under Accession number 53103. LGG bacteria were inoculated from a glycerol stock into Mann-Rogosa Sharpe (MRS) broth and cultured overnight under aerobic conditions at 37° C. without supplemental CO₂. The bacteria were centrifuged and washed twice with serum-free Dulbecco's Modified Eagle's Media (DMEM, neutral pH 7.1) to remove all MRS broth from the culture. The washed bacteria were inoculated into fresh serum-free DMEM at neutral pH 7.0-7.1 at a density of 0.5 OD₆₀₀, and cultured for an additional eight to 24 hours under aerobic conditions at 37° C. without supplemental CO₂. The culture was centrifuged to pellet the bacteria. The conditioned culture supernatant was separated from the bacterial pellet and was adjusted to neutral pH 7.1 with NaOH. Conditioned supernatant from L. rhamnosus GG cultures as well as other Lactobacilli including L. acidophilus (ATCC 4356), L. jensenii (ATCC 25258), L. plantarum (ATCC 14917), L. gasseri (ATCC 9857) and L. crispatus (ATCC 33197), and bacteria within the genera of Bifidobacterium, Streptococcus and Enterococcus (e.g., E. faecalis, ATCC 700802) indicate that these bacteria produce soluble factors that reduce HIV-1 replication in vitro.

Reverse Phase HPLC/C18 Fractionation. Conditioned supernatants from L. rhamnosus GG cultures were evaporated to dryness in a SPEED-VAC concentrator under dry ice. The dried pellet was resuspended in a small volume of H₂O/0.1% TFA and loaded onto a C18 reverse-phase column. The initial flow-through fraction was collected, while all other compounds retained on the column were removed. The flow-through fraction contained salts, hydrophilic amino acids and hydrophilic small peptides (<1000 Da)

SEPHADEX G-10 Fractionation. The flow-through fraction of the reverse phase column was de-salted and the low molecular weight compounds (<300 Da) were removed by size exclusion chromatography on a SEPHADEX G-10 column.

Mass Spectrometry and Edman's Sequencing. G-10 fractions were analyzed by Mass Spectrometry using MALDI-TOF and demonstrated a pattern of distinct peaks in the range of 500 Da to 700 Da. Edman's sequencing demonstrated a pattern of hydrophilic amino acids consistent with small peptides of four to six amino acid residues in length. Hydrophilic amino acids identified included, Aspartic Acid (D), Glutamic Acid (E), Arginine (R), Hisitidine (H), Serine (S), Threonine (T) and Glutamine (Q).

### EXAMPLE 2

### Characterization of Isolated Bioactive Molecules

Fractions containing bioactive small molecules were screened for activity on human cells of vaginal, cervical, intestinal, mammary, and lymphoid origin. Specifically, bioactivity and HIV-inhibition were evaluated in one or more of the following human cells, cell lines and tissues: CD4+ cells (TZM-b1), primary CD4+ lymphocytes, primary cervicovaginal tissues, mammary epithelial cells (MFC-10A), and intestinal epithelial cells (Caco-2).

Inhibition of HIV Replication. TZM-b1 cells are genetically engineered human cells that are highly susceptible to HIV-1 infection. These cells were treated with individual SEPHADEX G-10 fractions and infected with HIV-1. HIV inhibition was determined relative to media controls, and active fractions containing HIV-inhibitory activity were identified. The active fractions were evaluated by MALDI-TOF and demonstrated the presence of small molecules in the range of 500-700 Da.

Mechanism of Action Against HIV. HIV inhibition was evaluated by culture and PCR methods to determine whether the bioactive molecules block the early stages of the virus life cycle. Infection and replication of HIV-1 in activated CD4⁺ T lymphocytes, the primary target cell for HIV-1 in vivo, was evaluated. No significant effect was observed for HIV binding, entry, reverse transcription or integration when exposed to the bioactive molecules. Treatment was not associated with inhibition of viral binding to the target cell (absorption) via the envelope glycoproteins. Stimulation of integrated HIV-1 through the virus-long-terminal repeat (LTR) region indicated a reduction in expression at the level of virus transcription (post-integration). This finding does not preclude additional effects on later stages of the virus life cycle. Taken together, these results indicate that the bioactive small molecules produced by probiotic bacteria act to reduce expression of HIV-1 once the virus is integrated into the host cell, most likely through modulation of host cell pathways involved in regulating viral transcription.

Effect on Human Immune Mediators. The effect of bioactive small molecules was tested in human cells and tissues for the ability to modulate expression of host immune mediators. Cultures of human cells or primary tissue explants were treated for 48 hours with SEPHADEX G-10 fractions containing bioactive small molecules (500-700 Da), or appropriate controls, and the culture supernatants were tested in multiplex LUMINEX assays against a panel of 27 different human cytokines, chemokines and growth factors. Modulation in expression of human cytokines and chemokines, including, for example, IL-1ra, IL-6, IL-8, MCP-1, and IP-10, was noted for treated cervicovaginal tissues, and epithelial cells of mammary origin (MFC-10A). Modulation of cytokine and chemokine expression in human intestinal epithelial cells (Caco-2 cells) and primary human lymphocytes was also conducted to demonstrate the effects of the small bioactive molecules on these cell types. SEPHADEX G-10 fractions that decreased expression of immune mediators also decreased HIV-1 infection in parallel assays, suggesting a common mechanism of action involving host cell pathways that regulate both HIV transcription and expression of immune mediators.

Decreased Expression of Human Vasoendothelial Growth Factor (VEGF). LUMINEX assays revealed a decrease in expression of human VEGF in primary human cervicovaginal tissues treated with SEPHADEX G-10 fractions containing small bioactive molecules of 500-700 Da. The levels of VEGF secreted into the culture supernatants were significantly reduced (≧10-fold) in some of the treated tissues as compared to untreated controls. Similar results were obtained using human mammary epithelial cells (MFC-10A). The same SEPHADEX G-10 fractions that decreased VEGF expression also decreased HIV-1 replication, and down-regulated immune mediators (e.g., IL-1ra, IL-6, IL-8), thereby suggesting a common cellular signaling pathway involved in regulation of both HIV transcription and expression of host growth factors such as VEGF.

Decreased Activation of Cellular MAPK (Erk1/2) Pathways. In further experiments, CD4+ TZM-b1 cells were stimulated with serum to activate cellular MAPK pathways, either in the presence or absence of SEPHADEX G-10 fractions containing small bioactive molecules of 500-700 Da. Phosphorylation of Erk1/2 was detected in cell lysates by western blot analysis. Decreased Erk1/2 phosphorylation was demonstrated as early as five minutes after serum stimulation in cells treated with fractions containing small bioactive molecules of 500-700 Da as compared to control cells treated with media alone. These results indicate that the bioactive small molecules disclosed herein may decrease both HIV replication and host cell activation by inhibiting induction of this kinase pathway. Thus, the bioactive molecules do not appear to significantly interact with the virus itself, but rather act on the target cell to limit virus replication.

Biophysical Properties. Small bioactive molecules isolated from the probiotic bacteria disclosed herein were treated with either heat (99° C. for 15 minutes), protease digestion (trypsin, protease each at 100 μg/ml), or adjusted to pH 3.0-10.0 and then back to neutral pH, and were found to retain biological activity against HIV-1 under each of these conditions. Sensitivity to additional proteolytic enzymes including Proteinase K and pronase will indicate whether the small bioactive molecules are generally resistant to all proteases.

1. A method for isolating a bioactive molecule from a probiotic bacterium comprising passing probiotic bacterium-conditioned media through a reverse-phase chromatography matrix to obtain hydrophilic molecules having a molecular weight of less than 1000 Daltons; and fractionating the molecules by size exclusion to isolate a bioactive molecule characterized as being hydrophilic; stable to at least 99° C., stable at pH from about 3.0 to about 10.0, resistant to protease digestion, and having a molecular weight of between about 500 Daltons and about 700 Daltons, wherein said isolated bioactive molecule inhibits replication of human immunodeficiency virus, decreases expression of inflammatory cytokines and chemokines, decreases expression of vasoendothelial growth factor, and decreases extracellular signal-regulated kinases 1/2 (Erk1/2) activation.

2. The method of claim 1, wherein the probiotic bacterium is selected from the genera of Lactobacillus, Streptococcus, Enterococcus and Bifidobacterium.

3. The method of claim 2, wherein the probiotic bacterium is selected from the group of L. rhamnosus, L. acidophilus, L. jensenii, L. plantarum, L. gasseri and L. crispatus.

4. The method of claim 2, wherein the probiotic bacterium is selected from the group of S. thermophilus and S. salivarius.

5. The method of claim 2, wherein the probiotic bacterium is E. faecalis.

6. The method of claim 2, wherein the probiotic bacterium is selected from the group of B. animalis, B. breve, B. infantis, B. lactis, and B. longum.

7. The method of claim 1, wherein the reverse-phase matrix bears alkyl groups ranging in length from 4 to 18 carbon atoms.

8. The method of claim 7, wherein the reverse-phase chromatography matrix is a C₁₈ matrix.

9. (canceled)

10. An isolated bioactive molecule from a probiotic bacterium, wherein the isolated bioactive molecule is characterized as being hydrophilic; stable to at least 99° C.; stable at a pH in the range of about 3.0 to about 10.0; resistant to protease digestion; and having a molecular weight of between about 500 Daltons and about 700 Daltons, and wherein said isolated bioactive molecule inhibits replication of human immunodeficiency virus, decreases expression of inflammatory cytokines and chemokines, decreases expression of vasoendothelial growth factor, and decreases extracellular signal-regulated kinases 1/2 (Erk1/2) activation.

11. A method for inhibiting replication of human immunodeficiency virus (HIV) comprising contacting HIV-uninfected or HIV-infected cells with the isolated bioactive molecule of claim 10 thereby inhibiting the infection or replication of HIV.

12. A method for decreasing expression of inflammatory cytokines and chemokines comprising contacting cells with the isolated bioactive molecule of claim 10 thereby decreasing expression of inflammatory cytokines and chemokines in the cells as compared to cells not contacted with the isolated bioactive molecule.

13. The method of claim 12, wherein the cytokines and chemokines are selected from the group of IL-1ra, IL-6 and IL-8.

14. A method for decreasing expression of vasoendothelial growth factor (VEGF) comprising contacting cells with the isolated bioactive molecule of claim 10 thereby decreasing expression of VEGF in the cells as compared to cells not contacted with the isolated bioactive molecule.

15. A method for decreasing extracellular signal-regulated kinases 1/2 (Erk1/Erk2) activation comprising contacting cells with the isolated bioactive molecule of claim 10 thereby decreasing Erk1/Erk2 activation in the cells as compared to cells not contacted with the isolated bioactive molecule.

16. The method of claim 11, wherein the cells are human epithelial cells, or cells of primary tissues which contain epithelial cells and CD4+ cells.

17. A pharmaceutical composition comprising the isolated bioactive molecule of claim 10 in admixture with a pharmaceutically acceptable carrier.

18. The pharmaceutical composition of claim 17, wherein said composition is formulated for oral, rectal, buccal, vaginal, parenteral, topical, intranasal, transdermal, intraarticular, intrathecal or inhalation administration.

19. A method for preventing mucosal transmission of human immunodeficiency virus (HIV) comprising administering to the mucosa of a subject an effective amount of the pharmaceutical composition of claim 17, thereby preventing mucosal transmission of HIV to the subject.

20. The method of claim 19, wherein the pharmaceutical composition is formulated in an acid-buffering gel or cream for topical administration to the skin, vaginal surface or gastrointestinal surface.

21. The method of claim 19, wherein the pharmaceutical composition is formulated for oral administration to an infant exposed to HIV-1 through breastfeeding.
